(19) United States
(12) Patent Application Publication
(10) Pub. No.: US 2017/0169565 A1
(43) Pub. Date: Jun. 15, 2017

(54) OPTICAL COHERENCE TOMOGRAPHY GLAUCOMA DETECTION BASED ON RETINAL VESSEL RELIEF HEIGHT

(71) Applicant: Oregon Health & Science University, Portland, OR (US)

(72) Inventors: David HUANG, Portland, OR (US); Ou TAN, Portland, OR (US)

(21) Appl. No.: 15/116,942

(22) PCT Filed: Feb. 24, 2015
(86) PCT No.: PCT/US2015/017269

§ 371 Date: Aug. 5, 2016

Related U.S. Application Data
(60) Provisional application No. 61/944,136, filed on Feb. 25, 2014.

(51) Int. Cl. G06T 7/00; G06T 7/60; A61B 3/12; A61B 3/10; A61B 3/00

(52) U.S. Cl. CPC G06T 7/0012; A61B 3/102; A61B 3/0025; A61B 3/1225; G06T 7/60; G06T 2207/30041; G06T 2207/10101; G06T 2207/30101

(57) ABSTRACT

Systems and techniques for detecting glaucoma in a subject based on retinal vessel relief height obtained from optical coherence tomography (OCT) image data are disclosed. In one example approach, a retinal vessel relief height relative to a retinal plane may be calculated from OCT image data and the presence or absence of a glaucoma condition may be determined based on the retinal vessel relief height.

### CROSS-REFERENCE TO RELATED APPLICATION

This application claims priority to U.S. Provisional Patent Application No. 61/944,136, filed Feb. 25, 2014 and titled “OPTICAL COHERENCE TOMOGRAPHY GLAUCOMA DETECTION BASED ON RETINAL VESSEL RELIEF HEIGHT,” the entire contents of which are incorporated by reference herein.

### FIELD

The present disclosure relates to the field of optic neuropathic disease diagnosis, and, more specifically, to methods for detecting glaucoma using optical coherence tomography.

### BACKGROUND

Glaucoma is the leading cause of irreversible blindness worldwide. Glaucomatous optic neuropathy produces stereotypical optic nerve head (ONH) changes and results in corresponding patterns of vision loss, as well as structural changes in the retinal nerve fiber layer (RNFL) and ganglion cell complex (GCC). Therefore, rapid, objective, and reliable screening tools for the detection of glaucoma would be a major advance in the care of patients with glaucoma.

Optical coherence tomography (OCT) is a non-contact, non-invasive imaging modality for high-resolution, depth-resolved, cross-sectional, and three-dimensional (3D) imaging of biological tissue. Among its many applications, ocular imaging in particular has found widespread clinical use and can be performed quickly and easily with minimal expertise. As one example approach, retinal nerve fiber layer changes and ganglion cell complex loss, as determined from OCT image data, may be used to assist in detecting and diagnosing optic neuropathic diseases such as glaucoma. For example, approaches are known that attempt to detect and diagnose glaucoma based on measurements of the thickness of the RNFL and/or GCC as obtained from OCT image data.

However, due to the wide variability in ONH anatomy and RNFL thickness in the normal population, e.g., due to inborn variations, such approaches provide results that are subjective, inadequate, and unreliable for glaucoma detection. Therefore, in such approaches, OCT technology cannot be used as a reliable stand-alone glaucoma screening tool. Further, such approaches yield low sensitivity for the detection of early glaucoma since changes in RNFL and/or GCC may not be sufficiently pronounced during early stages of glaucoma.

### SUMMARY

Discussed herein are systems and techniques for detecting glaucoma in a subject based on retinal vessel relief height obtained from OCT image data. In one example approach, a retinal vessel relief height relative to a retinal plane may be calculated from OCT image data, and the presence or absence of a glaucoma condition may be determined based on the retinal vessel relief height.

As disclosed herein, OCT technology may be used to measure a retinal vessel relief height, e.g., a height of one or more retinal blood vessels relative to a nerve fiber layer surface, to indicate nerve fiber loss in a manner that is not affected by inborn variation in the nerve fiber layer thickness. For example, OCT-derived vessel relief height may be used alone as a glaucoma diagnostic. Such an approach may enable a more reliable, objective, and quantifiable early glaucoma diagnosis test that is non-invasive in nature.

Further, since vessel relief height may be more sensitive for early glaucoma diagnosis while retinal nerve fiber layer (RNFL) thickness may be more sensitive in advanced glaucoma, such an approach may be combined with RNFL thickness measurements obtained from OCT ocular imaging data in order to enhance detection sensitivity without significant loss of specificity. In some embodiments, this approach may provide a stand-alone OCT-based diagnostic.

Since OCT ocular imaging has found widespread clinical use and can be performed quickly and easily with minimal expertise, various ones of the embodiments disclosed herein may be readily adopted by practitioners to provide rapid, objective, and reliable screening tools for the detection of glaucoma in subjects, may be well-suited, for example, for screening undiagnosed, high risk individuals in periodic eye exams and/or for treatment monitoring (e.g., in individuals diagnosed with early to moderate glaucoma.

The foregoing Summary is provided to introduce a selection of concepts in a simplified form that are further described below in the Detailed Description. This Summary is not intended to identify key features or essential features of the subject matter disclosed herein, nor is it intended to be used to limit the scope of the subject matter disclosed herein. Furthermore, the embodiments disclosed herein are not limited to implementations that address any or all disadvantages noted in any part of this disclosure.

### BRIEF DESCRIPTION OF THE DRAWINGS

FIG. 1 shows example optical coherence tomography imaging and measurement of retinal vessel relief height and diameter.

FIG. 2 shows an example box-and-whisker plot analysis of retinal vessel relief height in control and glaucomatous eyes at various radii from the optic disc.

FIG. 3 shows an example Venn diagram analysis of retinal nerve fiber layer thickness versus retinal vessel relief height in control and glaucomatous eyes.

FIG. 4 shows an example scatter plot analysis of retinal vessel relief height versus known structural and functional parameters in control and glaucomatous eyes.

FIG. 5 shows an example box-and-whisker plot analysis of retinal vessel diameter in control and glaucomatous eyes at various radii from the optic disc.

FIG. 6 shows an example scatter plot analysis of retinal vessel diameter versus known structural and functional parameters in control and glaucomatous eyes.

FIG. 7 shows (A) an example of an image of a glaucomatous eye with significant nerve fiber layer thinning with normal vessel relief height and (B) an example of an image of a glaucomatous eye with normal nerve fiber layer thickness and significantly increased vessel relief height.

FIG. 8 shows an example flow diagram for a method for detecting optic neuropathy in a subject in accordance with various embodiments disclosed herein.

FIG. 9 schematically shows an example computing system in accordance with various embodiments disclosed herein.

FIG. 10 shows an example flow diagram for a method of automatic vessel relief height estimation in accordance with various embodiments disclosed herein.

FIG. 11 shows example illustrations of various operations in the method of FIG. 10 in accordance with various embodiments disclosed herein.

### DETAILED DESCRIPTION

Various embodiments discussed in the following detailed description are directed to detecting optic neuropathy in a subject based on retinal vessel relief height measurements obtained from optical coherence tomography (OCT) image data. In the following detailed description, reference is made to the accompanying drawings which form a part hereof, and in which are shown, by way of illustration, embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

Optical coherence tomography (OCT) is an optical signal acquisition and processing method that is capable of capturing micrometer-resolution, three-dimensional images of optical scattering media, e.g., biological tissue. Optical coherence tomography is based on interferometric techniques and typically employs near-infrared light. The use of relatively long wavelength light allows it to penetrate into the scattering medium. As remarked above, among its many applications, OCT-based ocular imaging has found widespread clinical use and can be performed quickly and easily with minimal expertise. OCT is a non-invasive imaging modality that can provide accurate and precise anatomical measurements of the optic nerve and retinal layers for use in detecting and diagnosing optic neuropathic diseases, such as glaucoma, in accordance with various ones of the embodiments disclosed herein.

For example, retinal nerve fiber layer changes and ganglion cell complex loss, as determined from OCT image data, may be used to assist in detecting and diagnosing optic neuropathic diseases such as glaucoma. In particular, approaches are known that attempt to detect and diagnose glaucoma based on measurements of the thickness of the retinal nerve fiber layer (RNFL) and/or ganglion cell complex (GCC) as obtained from OCT image data. However, due to the wide variability in optic nerve head (ONH) anatomy and RNFL thickness in the normal population, e.g., due to inborn variations, such approaches provide results that are subjective, inadequate, and unreliable for glaucoma detection. Therefore, in such approaches, OCT technology cannot be used as a reliable stand-alone glaucoma screening tool. Further, such approaches yield low sensitivity for the detection of early glaucoma since changes in RNFL and/or GCC may not be sufficiently pronounced during early stages of glaucoma.

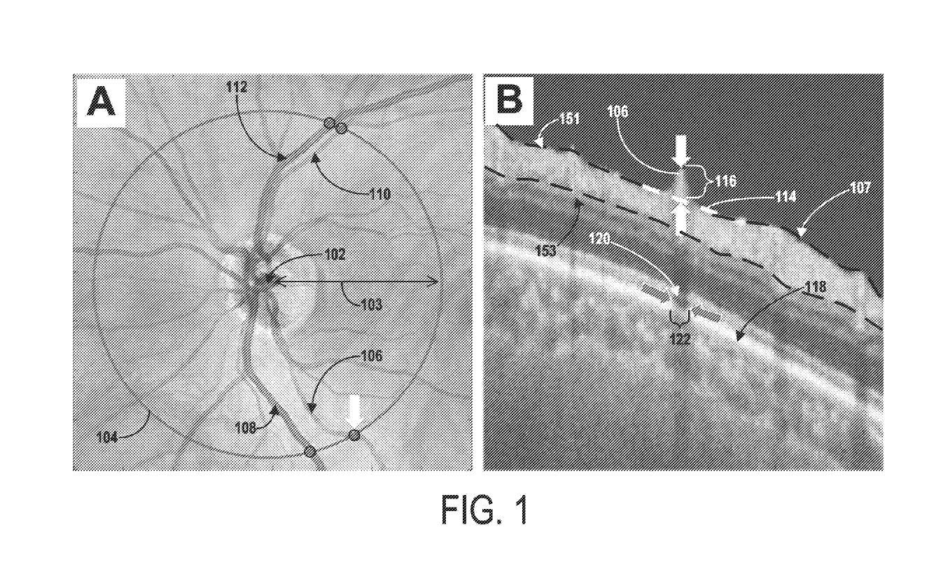

The inventors herein have recognized a correlation between retinal vessel relief height and glaucoma, and have developed diagnostics using particular OCT techniques that build on this correlation. As used herein, the term “retinal vessel relief height” is used to refer to either a height of a retinal blood vessel relative to a plane of the retina or an average height relative to the retinal plane of one or more retinal blood vessels, e.g., one or more of the superior and inferior temporal arcade arteries and veins. For example, FIG. 1 shows example OCT images and illustrates a measurement of a retinal vessel relief height of a retinal blood vessel 106. In particular, FIG. 1A shows an example OCT image centered on the ONH (or optic disc) 102. As used herein, the term “optic disc/optic nerve head” is used to refer to the location where ganglion cell axons exit the eye to form the optic nerve. A circular scan at a radius 103 from the center of the optic disc 102 is shown bounded by the circle labeled 104. The intersections of the four major retinal vessels, the superior and inferior temporal arcade arteries and veins 106, 108, 110 and 112, are shown encircled in FIG. 1A within the boundary circle 104 having the radius 103. The intersection of vessel 106 with the boundary circle 104 at the radius 103 from the center of the optic disc 102 is depicted in FIG. 1B as part of a representative cross-sectional scan of retinal vessel 106 and underlying retina 107. In FIG. 1B, the vessel relief height 116 is depicted between the white arrows as the distance between vessel 106 and the retinal plane 114 (depicted by a dashed line) along a direction substantially normal to the inner surface of retina 107 at the intersection of vessel 106 with the boundary circle 104.

This vessel relief height 116 at the radius 103 may be calculated for one or more retinal vessels, e.g., the vessel relief height may be calculated for all of the four major retinal blood vessels 106, 108, 110, 112, and averaged to obtain a vessel relief height value that may be used in the detection of the presence or absence of an optic neuropathic disease such as glaucoma. For example, if the vessel relief height of one or more blood vessels at a predetermined radius (or a vessel relief height value based on the vessel relief height of one or more individual blood vessels) is found to be greater than a vessel relief height threshold, then an optic neuropathic disease such as glaucoma may be indicated. The vessel relief height threshold may be based on any suitable data, e.g., the threshold may be predetermined and may be based on vessel relief height data obtained from eyes of subjects who lack optic neuropathic disease, e.g., data from a normal control population.

As described above with regard to FIG. 1, the calculation of a vessel relief height is obtained from a circular OCT scan at a predetermined radius 103 from the center of the optic disc 102 of the eye of a subject. The radius 103 at which the vessel relief height is measured may be any suitable radius. However, the inventors herein have unexpectedly found the optimal radius at which to calculate vessel relief height for glaucoma detection to be approximately 3.0 mm from the center of the optic disc 102. In particular, since glaucomatous RNFL thickness change was previously assumed to be greatest at smaller radii from the optic disc center, it was expected that one would find a greater change in retinal vessel relief height at smaller radii. However, based on measurements of vessel relief height at different radii from the optic disc center as described in more detail below, the greatest glaucomatous vessel relief height changes were unexpectedly found at a radius of 3.0 mm from the optic disc center.

The inventors herein have further recognized that such vessel relief height measurements may be used to complement OCT-derived RNFL thickness measurements in order to achieve substantial enhancement of detection sensitivity without significant loss of specificity in the diagnosis of glaucoma. For example, retinal vessel relief height may be less variable in normal eyes and better suited to detecting early glaucoma as compared to RNFL thickness. Thus, when combined together, RNFL and vessel relief height measurements may enable increased sensitivity of detection of the full range of glaucoma severity. For example, in response to determining an absence of an optic neuropathic disease based on the retinal vessel relief height, a retinal nerve fiber layer thickness may be calculated from the OCT image and used to determine the presence or absence of an optic neuropathic disease. Further, the presence of an early stage optic neuropathic disease may be identified in response to determining that a retinal vessel relief height is greater than a retinal vessel relief height threshold and the presence of an advanced stage optic neuropathic disease may be identified in response to determining that a retinal nerve fiber layer thickness is less than a retinal nerve fiber layer thickness threshold.

### Example

The example discussed below describes a pilot study of OCT measurements of retinal vessel relief heights and diameters in eyes of control participants and in participants with preperimetric glaucoma (PPG) and perimetric glaucoma (PG) and demonstrates the use of vessel relief height measurements in detecting the presence or absence of an optic neuropathic disease such a glaucoma, in accordance with various embodiments. In this example, OCT image data were obtained from circular OCT scans, processing was performed on the OCT data, and parameters such as vessel relief height, vessel diameter, and RNFL thickness were extracted from the OCT data and used to detect the presence or absence of glaucoma. Embodiments may vary as to the methods of obtaining OCT image data, performing OCT data processing (e.g., using various conventional image processing techniques), and extracting parameters from the OCT data. For example, different embodiments may utilize different types of OCT, speeds of OCT, scan patterns, eye motion correction techniques, and/or frame averaging techniques. Further, though the example illustrates applications of OCT technology to glaucoma detection, such an approach may be used for the detection of other optic neuropathic diseases or ocular disorders. For example, diseases that cause RNFL damage may also cause vessel relief distortion, and vessel relief height measurements may detect or assist in detecting these diseases. The distortion patterns themselves may vary (e.g., if a disease only causes superior RNFL damage, the vessel relief height may only be abnormal in the superior region). The example discussed below is for illustrative purposes and is not intended to be limiting.

Materials and Methods: The research protocol used in this example was approved by the institutional review boards at Oregon Health and Science University (OHSU) and carried out in accordance with the tenets of the Declaration of Helsinki. Written informed consent was obtained from each subject after explanation of the nature of the study. Participants were recruited at the Casey Eye Institute/OHSU according to the Advanced Imaging for Glaucoma (AIG) study protocol. The OHSU ancillary site followed the same eligibility and endpoint protocol as the AIG study, but used an advanced 100 kHz swept-source OCT system instead of commercially available advanced imaging instruments (e.g., a 27 kHz spectral OCT). The advanced swept-source OCT system, which was faster than a 27 kHz spectral OCT, also utilized an orthogonal registration algorithm to reduce eye motion artifacts in volumetric scans. The inclusion and exclusion criteria of the AIG study is described in Tan et al. 2009 (Tan O, Chopra V, Lu A T, Schuman J S, Ishikawa H, Wollstein G, Varma R, Huang D. Detection of macular ganglion cell loss in glaucoma by Fourier-domain optical coherence tomography. Ophthalmology. 2009 December; 116(12):2305-14.e1-2). In particular, normal control participants met the following criteria in both eyes: intraocular pressures (TOP) of less than 21 millimeters Hg for both eyes, a normal Humphrey visual field (HVF) on achromatic standard automated perimetry by Swedish Interactive Threshold Algorithm 24-2 (HFA II; Carl Zeiss Meditec, Inc., Dublin, Calif.) with mean deviation (MD), Glaucoma Hemifield Test (GHT), and pattern standard deviation (PSD) within normal limits. In addition, normal subjects had a normal appearing ONH and RNFL on ophthalmoscopic examination, and an open angle by gonioscopy. The inclusion criteria for PG participants included at least one eye that fulfilled the following criteria: 1) glaucomatous HVF with PSD or GHT outside normal limits (P<0.05 and P<1%, respectively) in a consistent pattern on both qualifying HVF, and 2) glaucomatous ONH or RNFL defects. The PPG participant group did not have glaucomatous HVF as defined for the PG group, but had glaucomatous ONH or RNFL defects. Exclusion criteria for all groups included vision less than 20/40, age less than 40 or greater than 79 years at enrollment, any ocular surgery other than cataract extraction, other diseases that might cause HVF or ONH abnormality, and factors that might preclude the participant from performing study procedures or complete the study.

Optical Coherence Tomography and Image Analysis: Unlike other AIG clinical centers, which use commercial time-domain and Fourier-domain OCT systems, in this example a prototype high speed swept-source Fourier-domain OCT system was used. The prototype was built by the Laser Medicine and Medical Imaging Group at the Massachusetts Institute of Technology and followed the configuration described in Potsaid et al. 2010 (Potsaid B, Baumann B, Huang D, Barry S, Cable A E, Schuman J S, Duker J S, Fujimoto J G. Ultrahigh speed 1050 nm swept source/Fourier domain OCT retinal and anterior segment imaging at 100,000 to 400,000 axial scans per second. Opt Express. 2010 Sep. 13; 18(19):20029-48). The device operated at an axial scan speed of 100 kHz using a swept source cavity laser operating at 1050 nm with a tuning range of 100 nm. A resolution of 5.3 μm axially and 18 μm laterally at an imaging depth of 2.9 mm in tissue was achieved. The ocular light power exposure was 1.9 mW, which was within the American National Standards Institute (ANSI) safety limit. It should be understood that the OCT system, OCT scanning parameters, and image analysis protocols described in this example is provided for illustrative purposes and is not intended to be limiting. In particular, any suitable OCT system, parameters, and image analysis protocols may be used without departing from the scope of this disclosure. For example, any OCT system that can make circular or volumetric scans may be suitable, and faster OCT systems may provide improved results due to less eye motion artifact.

Scan Pattern: Participants were scanned using a high density raster scan. The scan covered an 8 mm×8 mm area, centered at the ONH. In the fast transverse scan direction, the B-scan consisted of 640 A-scans. The slow transverse scan direction included 640 B-scans. The entire raster scan was obtained in approximately 4 seconds. In the scan protocol, one eye of each participant was scanned with 4 scans consisting of 2 horizontal and 2 vertical scans. It should be understood that the OCT scanning protocol described in this example is provided for illustrative purposes and is not intended to be limiting. In particular, any suitable OCT scanning protocol with any suitable number of scans in any suitable time frames may be used without departing from the scope of this disclosure. For example, a circular scan resampled from volumetric data and averaged over multiple scans may be used, as discussed below. In other examples, a simple circular scan may be used. Improved results may be obtained, however, from the use of a circular scan with high spatial density, less motion artifact, and well-centered on the optic disc.

Three-Dimensional Orthogonal Registration: In order to reduce eye motion artifact during scanning, an orthogonal registration algorithm was applied to register all 4 scans as described in Kraus et al. 2012 (Kraus M F, Potsaid B, Mayer M A, Bock R, Baumann B, Liu J J, Hornegger J, Fujimoto J G. Motion correction in optical coherence tomography volumes on a per A-scan basis using orthogonal scan patterns. Biomed Opt Express. 2012 Jun. 1; 3(6):1182-99). In this algorithm, multiple successive volume scans and orthogonal fast scans were registered retrospectively in order to estimate and correct for eye motion. The algorithm estimated motion by iteratively optimizing a global objective function to make all volumes similar and penalizing modeled motion within a short time scale. The motion estimates were used to construct registered volumes without eye motion. These volumes were then combined into a single merged volume using an adaptive weighted sum. It should be understood that the registration algorithm described in this example is provided for illustrative purposes and is not intended to be limiting. In particular, any suitable registration algorithm capable of sufficiently removing eye motion artifact may be applied to OCT scans without departing from the scope of this disclosure. Further, in some embodiments, OCT scan registration may be omitted (e.g., when the amount of eye motion artifact is minimal or zero due to the use of a fast, single circular scan).

Image Processing: The merged volume scan was averaged along the depth direction of each eye to create a projection view of the ONH. In this example, the ONH center was manually selected. However, in some embodiments, the ONH center may be automatically identified and selected in any suitable way. For example, the retina pigment epithelium tips around the optic disc can be used for automatic disc boundary detection and the center can be used as disc center. Concentric rings of varying radii were measured and circular scans were resampled from the volume scan based on interpolation. Four major retinal vessels (the superior and inferior temporal arcade arteries and veins) were labeled on the enface view and their position on each circular scan was recorded. Vessel relief height was determined by measuring the number of pixels corresponding to the portion of the vessel above the retinal plane, followed by conversion to micrometers. Vessel diameter was calculated in a similar fashion using the vessel signal shadow cast upon the outer retinal layers. Vessel relief height and diameter were graded for the 4 identified vessels on each OCT circular scan image and averaged from four vessels for each circular scan. In this example, the anterior retinal plane, vessel peaks, and vessel shadow locations were manually selected using Logger Pro software (Vernier, Beverton, Oreg.). However, in some embodiments, one or more of these locations may be automatically identified and selected in any suitable manner. For example, the transverse position of a vessel along a circle can be detected based on the shadow on the retinal pigment epithelieum (RPE) band in the cross section images (e.g., see FIG. 1B). Further, it should be understood that though vessel relief height and diameter measurements were performed manually in this example, in some embodiments an automated measurement program may be used to automatically determine vessel relief height and diameter from OCT image data in order to standardize measurement parameters and reduce inter- and intra-observer variability. Automation of anatomical location identification and determination of anatomical dimensions, such as vessel relief height, vessel diameter, RNFL thickness, etc. may be performed in any suitable manner. For example with reference to FIG. 1, first the inner limited membrane (ILM) 151 and nerve fiber layer (NFL) boundary 153 may be automatically detected on two-dimensional (2D) or 3D OCT images. The vessel relief height can be calculated using the distance from the ILM position at the center of a vessel to the average ILM position on the neighbor axial scans of the same vessel. Vessel diameter may be calculated as the width of the shadow area on the RPE band 118. RNFL thickness may be calculated as the distance from the ILM 151 to the NFL boundary 153. Further, various processing algorithms may be performed in order to identify and/or reduce error in measurements obtained from OCT image data. For example, the use of circular OCT scans may introduce a small measurement error of vessel diameter, as the circular scan results in a slightly oblique (rather than truly perpendicular) cross-section of the vessel in the final 2D image used for vessel diameter measurement. Thus, processing may be performed in order to account for this error (e.g., by estimating the angle between the vessel and the scan circles based on an en face projection, and using the estimate to correct the error).

An OCT scan with a standard radius of 1.7 mm was resampled from the volume scan and automated NFL segmentation was developed for RNFL thickness on this circular scan. The RNFL thickness profile was defined as the distance between the ILM 151 and the lower RNFL boundary 153, and RNFL thickness was determined by averaging along the circular scan. The algorithm used was similar to other NFL thickness detection algorithms and automated software used in commercial OCT. One example of RNFL detection is described in Alasil et al. 2008 (Alasil T, Tan O, Lu A T, Huang D, Sadun A A. Correlation of Fourier domain optical coherence tomography retinal nerve fiber layer maps with visual fields in nonarteritic ischemic optic neuropathy. Ophthalmic Surg Lasers Imaging. 2008 July-August; 39(4 Suppl):S71-9), the entirety of which is hereby incorporated by reference. In the Alasil reference, the internal limiting membrane and inner segment and outer segment (IS/OS) junction were detected as the first and second gradient peak, the outer nerve fiber layer boundary was detected as the last gradient peak in front of the second white band in the usual intensity distribution pattern between the internal limiting membrane and IS/OS junction, and the peripapillary nerve fiber layer thickness was calculated as equal to the distance from the internal limiting membrane to the outer nerve fiber layer boundary. It should be understood that the image processing and parameter extraction routines described in this example are provided for illustrative purposes and are not intended to be limiting. Embodiments may vary as to the methods for performing OCT data processing, and extracting parameters such as vessel relief height, vessel diameter, and nerve fiber layer thickness from the OCT data.

Statistical Analysis: The Wilcoxon signed-rank test was performed to determine statistical significance between the study groups. The pooled standard deviation of repeat measurements and intraclass correlation were used to assess repeatability. The area under the receiver operating characteristic curve (AROC) was calculated for diagnostic power. The Pearson correlation was used for correlation between vessel relief height, RNFL, MD, and PSD.

Results: Forty-one participants were enrolled in the study between September, 2011 and May, 2013. Three participants were excluded due to weak OCT signal strength and 2 participants were excluded due to blurred images after orthogonal registration. The 36 remaining patients in the study comprised 25 healthy controls, 4 PPG, and 7 PG participants. These patients completed the study without any adverse effects. In the control group, the average age was 54±10 years, 68% were female, and the mean HVF MD was 0.19±0.78 dB. In the PPG and PG groups, the average age was 70±7 and 68±12 years old, 14% and 0% were female, and the mean HVF MD was 0.69±0.61 and −5.05±4.80, respectively.

After manual identification of the optic disc center and vessels as illustrated in FIG. 1A described above, vessel relief height and diameter were measured at various radii as described above and shown in FIG. 1B. In particular, FIG. 1A shows an enface OCT image centered on the optic disc 102 where the four major retinal vessels 106, 108, 110, and 112 are encircled at a boundary circle 104 of a circular scan at a radius of 3.5 mm. The vessel 106 marked by the white arrow is depicted in FIG. 1B as part of a representative cross-sectional scan of a major retinal vessel and the underlying retina 107. The vessel relief height 116 is depicted between the white arrows as a distance between the peak of the vessel 106 and the retinal plane 114 depicted by a dashed line. The vessel diameter 122 was calculated from the width of the shadow 120 cast by the vessel upon the outer retinal layers 118. Other non-peak features may be used to characterize the vessel relief height, such as the average height in the vessel area.

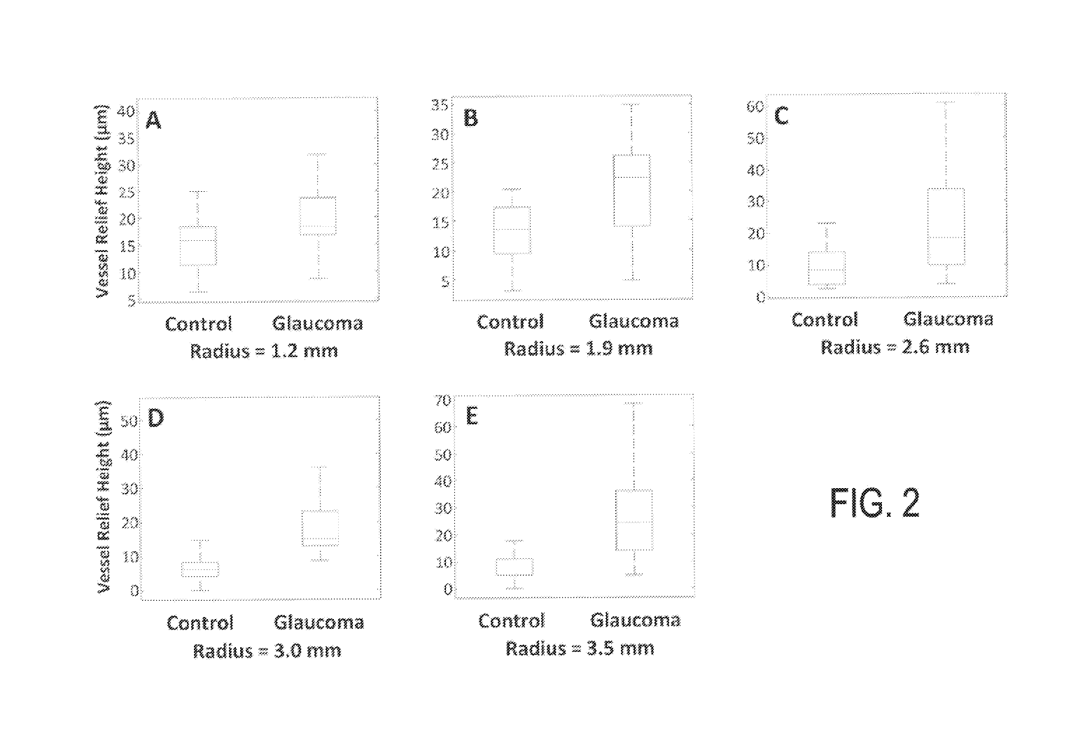

Table 1 shows vessel relief height data displayed as mean±standard deviation as a function of the radial distance from the optic disc center and FIG. 2 shows an example box-and-whisker plot analysis of retinal vessel relief height in control and glaucomatous eyes at various radii from the optic disc, where the lines within boxes represent the mean and the tails were calculated using the Wilcoxon signed-rank test. As demonstrated by the data shown in Table 1 and the plots shown in FIG. 2, vessel relief height was significantly larger in glaucoma eyes compared to control eyes at radii ranging from 1.9-3.5 mm from the optic disc center. The difference in vessel relief height between glaucoma and control eyes was most pronounced at a radius of 3.0 mm.

TABLE 1

Vessel Relief Height (μm)

| Radius (mm) | Control | Glaucoma | p Value |
|---|---|---|---|
| 1.2 | 15.1 ± 5.0 | 20.4 ± 9.7 | 0.064 |
| 1.9 | 13.1 ± 4.7 | 20.1 ± 8.7 | 0.013 |
| 2.6 | 9.4 ± 6.0 | 23.7 ± 17.5 | 0.007 |
| 3 | 7.1 ± 4.5 | 20.8 ± 13.4 | <0.001 |
| 3.5 | 8.4 ± 4.5 | 26.2 ± 17.7 | 0.001 |

The diagnostic power of vessel relief height measurements in the setting of glaucoma was then determined by measuring the AROC values at each radius. Table 2 shows AROC±standard error of the mean (SEM) for increasing radii and for RNFL thickness. As shown in Table 2, the diagnostic power of vessel relief height increased with larger radii and peaked at a radius of 3.0 mm. In addition, as shown in Table 2, at a radius of 3.0 mm the AROC value for vessel relief height was comparable to the AROC value for RNFL thickness in glaucoma diagnosis.

TABLE 2

| | Radius (mm) | AROC ± SEM |
|---|---|---|
| Vessel Height | 1.2 | 0.70 ± 0.11 |
| | 1.9 | 0.76 ± 0.11 |
| | 2.6 | 0.79 ± 0.09 |
| | 3.0 | 0.93 ± 0.04 |
| | 3.5 | 0.86 ± 0.08 |
| RNFL Thickness | 1.7 | 0.92 ± 0.05 |

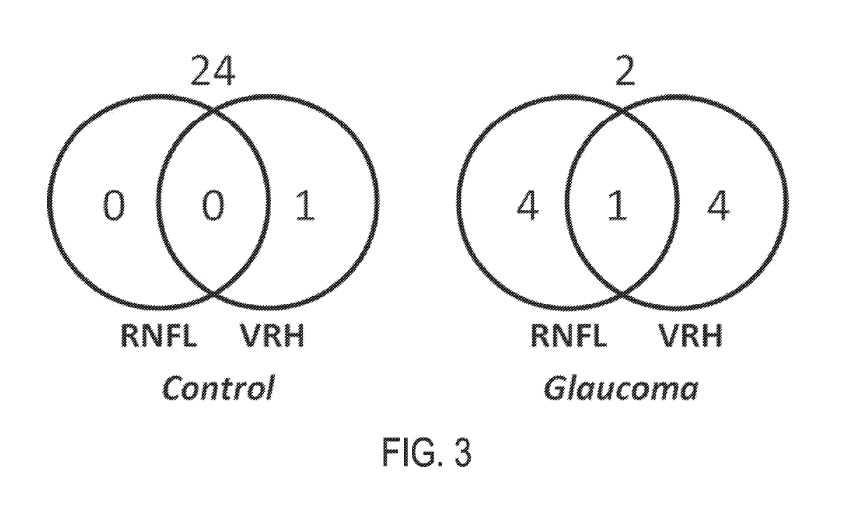

As remarked above, approaches are known that attempt to detect and diagnose glaucoma based on measurements of the thickness of the RNFL. However, due to the wide variability in ONH anatomy and RNFL thickness in the normal population, e.g., due to inborn variations, such approaches provide results that are subjective, inadequate, and unreliable for glaucoma detection. Therefore, in such approaches, OCT technology cannot be used as a reliable stand-alone glaucoma screening tool. Further, such approaches yield low sensitivity for the detection of early glaucoma since changes in RNFL and/or GCC may not be sufficiently pronounced during early stages of glaucoma. However, the combination of the vessel relief height and RNFL thickness may enhance the ability to diagnose glaucoma using OCT as a stand-alone diagnostic tool. For example, FIG. 3 shows a Venn diagram analysis of thickness (labeled “RNFL”) versus retinal vessel relief height (labeled “VRH”) in control and glaucomatous eyes. In this example, vessel relief height is measured at a radius of 3.0 mm from the optic disc, with a 99th percentile cutoff for vessel relief height (2.33 standard deviations above the mean of a healthy control group) and 1st percentile cutoff for RNFL (2.33 standard deviations below the mean). This Venn diagram analysis shows that the combination of vessel relief height (at a radius of 3.0 mm) and RNFL thickness increased the sensitivity of glaucoma diagnosis from 45% to 82%, and only slightly decreased specificity from 100% to 96%, relative to RNFL thickness alone.

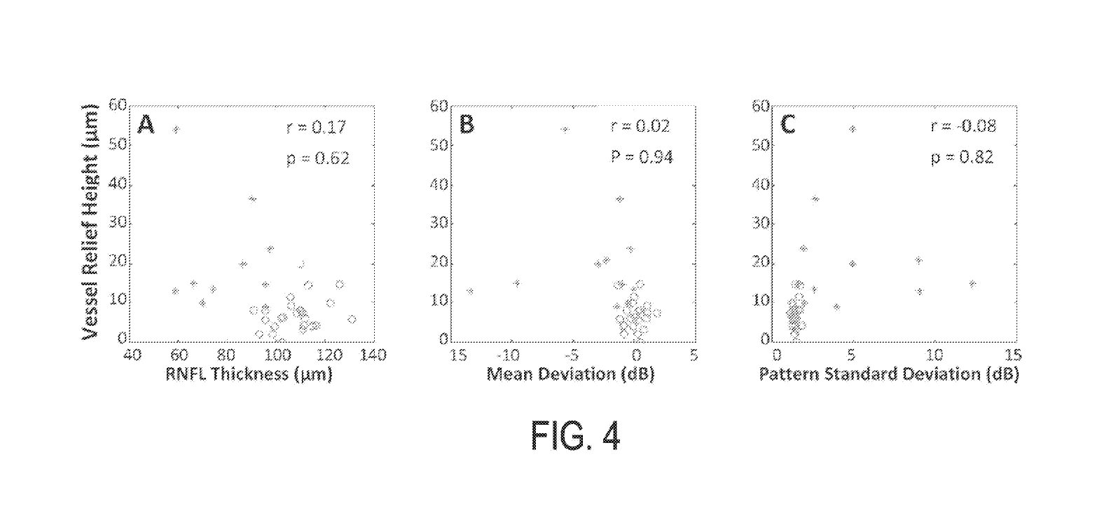

To elucidate possible correlations of vessel relief height with other quantitative measurements of glaucoma, vessel relief height was plotted versus RNFL thickness, mean deviation (MD), and pattern standard deviation (PSD) for each participant eye as shown in FIG. 4. FIG. 4 shows a scatter plot analysis of retinal vessel relief height versus known structural and functional parameters in control and glaucomatous eyes. In particular, FIG. 4A shows the retinal vessel relief height at a radius of 3.0 mm from the optic disc versus RNFL thickness, FIG. 4B shows the retinal vessel relief height at a radius of 3.0 mm from the optic disc versus MD, and FIG. 4C shows retinal vessel relief height at a radius of 3.0 mm from the optic disc versus PSD derived from standard automated perimetry. In FIG. 4, open circles shown in the plots indicate data from control eyes and asterisks indicate data from glaucomatous eyes.

For the combined group comprising PG and PPG participants, no statistically significant correlation was found between vessel relief height and RNFL thickness, MD, or PSD. The lack of correlation may be due to the nonlinear and non-monotonic relationship between vessel relief height and the other glaucoma diagnostic variables. Vessel relief height appears to increase in early glaucoma and then decline to near-normal levels in more advanced glaucoma as shown in FIG. 4.

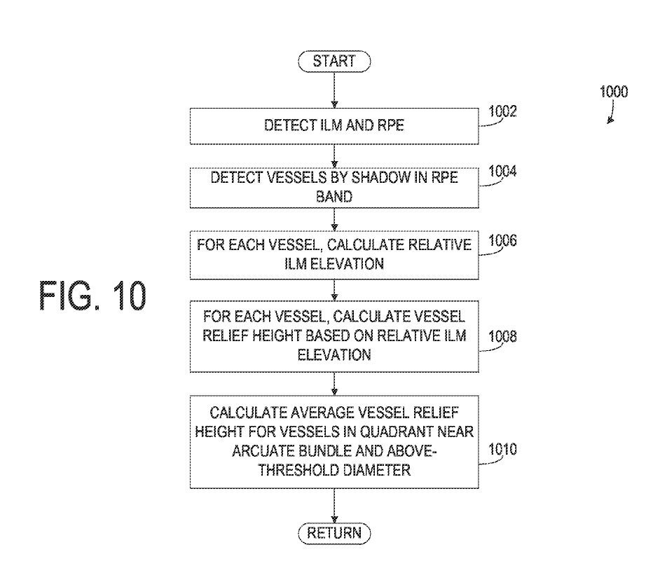

In this example, vessel relief height measurements were made manually, thus repeatability of vessel relief height measurements was determined in a masked fashion by a single observer. At a radius of 3.0 mm, the pooled standard deviation for vessel relief height was 2.38 μm and 4.01 μm, while intraclass correlation was 0.76 and 0.93 for control and glaucoma groups, respectively. It should be understood that, though this example describes manual calculation of vessel relief height from OCT data, in some embodiments vessel relief measurements from OCT image data may be performed automatically, e.g., via a computing device, as discussed below with reference to FIG. 10.

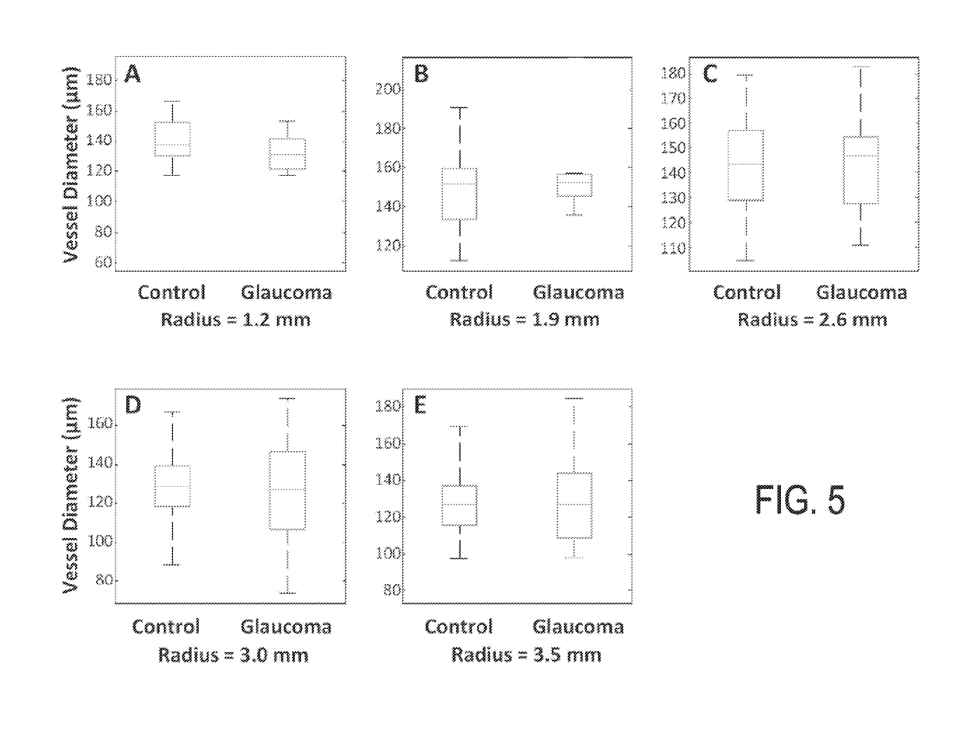

To determine whether retinal vessel diameter was also significantly different between control eyes and eyes with glaucoma, vessel diameters were measured at various radii as described above. FIG. 5 shows a box-and-whisker plot analysis of retinal vessel diameter in control and glaucomatous eyes at various radii from the optic disc, where the lines within the boxes represent the mean and the tails were calculated using the Wilcoxon signed-rank test. Table 3 shows vessel diameter displayed as mean±standard deviation as a function of radial distance from the optic disc center for control eyes and glaucomatous eyes, together with a p-value at each radius.

TABLE 3

Vessel Diameter (μm)

| Radius (mm) | Control | Glaucoma | p Value |
|---|---|---|---|
| 1.2 | 140 ± 23 | 133 ± 12 | 0.17 |
| 1.9 | 149 ± 18 | 157 ± 25 | 0.90 |
| 2.6 | 144 ± 20 | 144 ± 21 | 0.99 |
| 3.0 | 131 ± 20 | 124 ± 30 | 0.45 |
| 3.5 | 123 ± 20 | 135 ± 27 | 0.58 |

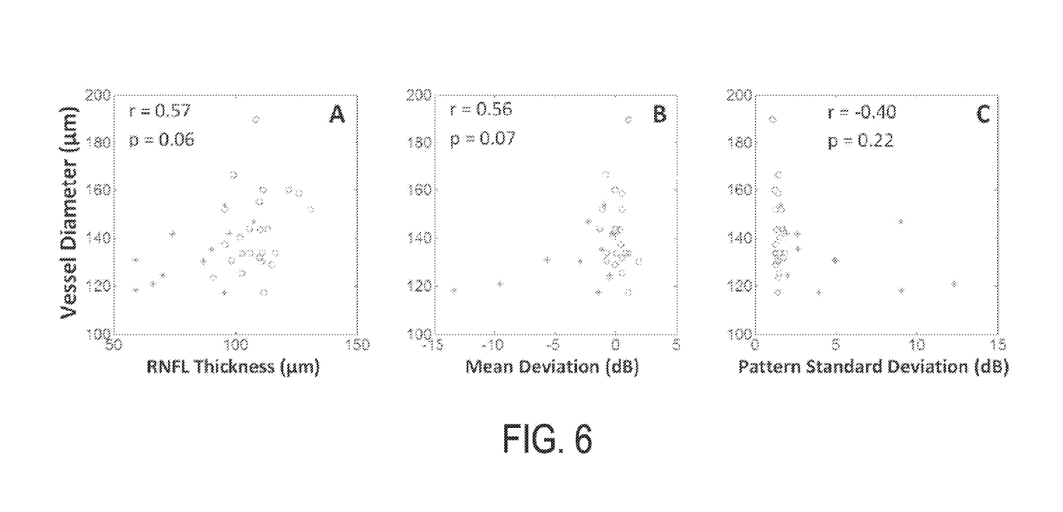

As shown in FIG. 5 and Table 3, no significant difference was noted in vessel diameter between control and glaucomatous eyes at any of the measured radii. However, as shown in FIG. 6, a scatter plot analysis of retinal vessel diameter as a function of RNFL, MD, and PSD suggests a trend of decreased retinal vessel diameter with increasing glaucoma severity. FIG. 6 shows a scatter plot analysis of retinal vessel diameter versus known structural and functional parameters in control and glaucomatous eyes. In particular, FIG. 6A shows retinal vessel diameter measurements at a radius of 3.0 mm versus RNFL thickness, FIG. 6B shows retinal vessel diameter measurements at a radius of 3.0 mm versus MD, and FIG. 6C shows retinal vessel diameter measurements at a radius of 3.0 mm versus PSD derived from standard automated perimetry. In FIG. 6, open circles indicate data from control eyes and asterisks indicate data from glaucomatous eyes.

The above example describes correlations between OCT-derived retinal vessel relief height and glaucoma and demonstrates that the diagnostic power of vessel relief height in the setting of glaucoma was comparable to RNFL thickness as measured by OCT. In addition, the example demonstrates a biphasic relationship between retinal vessel relief height and glaucoma severity and that vessel relief height measurements may be used to compliment RNFL thickness in the diagnosis of glaucoma with substantial enhancement of detection sensitivity without significant loss of specificity. For example, retinal vessel relief height may increase in early glaucoma and return to near normal levels with advanced glaucoma, as measured by both objective (e.g., RNFL thickness) and subjective (e.g., MD and PSD) parameters. This may be explained by the anatomic changes that occur with glaucoma. With early and moderate glaucoma, the inner retina may become thinner owing to loss of degenerating axons (reflected in decreased RNFL thickness) and loss of retinal ganglion cells (reflected in decreased ganglion cell layer complex thickness). As the major retinal vessels course through the inner retina, inner retinal thinning may result in retinal vessel exposure and increased relief height that can be measured using OCT. In addition to the retinal changes, a reduction in major retinal vessel diameter with increasing glaucoma severity may occur, e.g., as measured by RNFL thickness, MD, and PSD. Reduction in vessel caliber may in turn reduce the overall measured vessel relief height in moderate to severe glaucoma. Therefore, the largest vessel relief height measurements may be seen in early to moderate glaucoma.

The above example further demonstrates that OCT imaging may provide a stand-alone glaucoma diagnostic tool by using OCT-derived vessel relief height and OCT-derived RNFL thickness in the diagnosis of glaucoma. In particular, the example demonstrates that both retinal vessel relief height and RNFL measurements may be highly specific in ruling out glaucoma in healthy participants, as demonstrated by the Venn diagram analysis described above with regard to FIG. 3. Thus, combining RNFL thickness and retinal vessel height measurements may result in increased sensitivity for glaucoma detection. In the above example, this increased sensitivity was complimentary, as four out of six glaucoma patients would have been missed if only RNFL or retinal vessel height measurements were used alone.

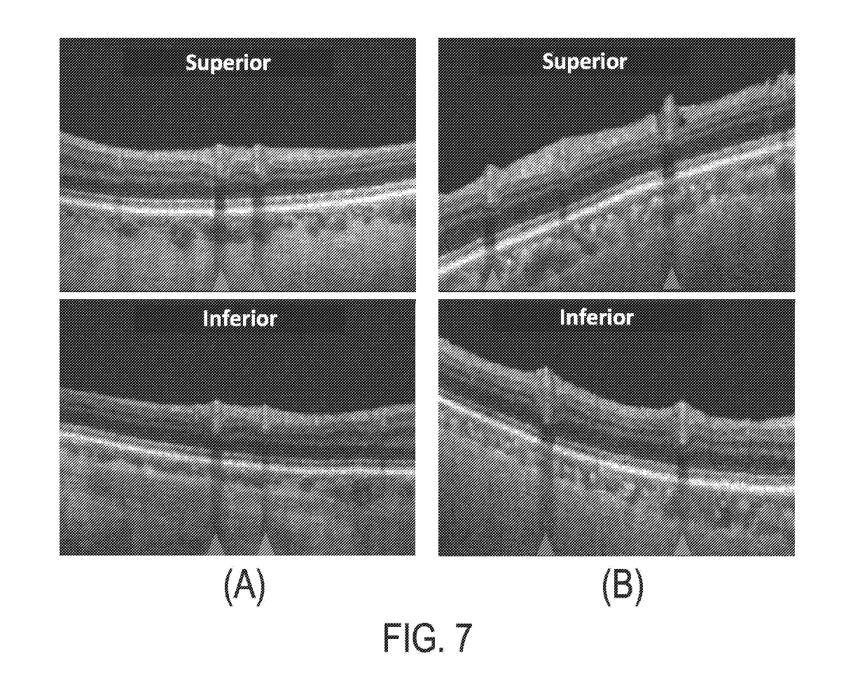

Representative OCT sections from these two types of cases are shown in FIG. 7. In particular, FIG. 7 shows (A) an example of an image of a glaucomatous eye with significant NFL thinning with normal vessel relief height and (B) an example of an image of a glaucomatous eye with normal NFL thickness and significantly increased vessel relief height. Arrowheads point to the arcuate vessels selected for the vessel relief height calculation. Vessel size may be smaller in the NFL-thinning-only eyes (121±30 μm) compared to vessel relief height—increase-only eyes (145±26 μm), while the NFL is thicker in vessel relief height—increase-only cases (95.4±9.3 μm) compared to NFL-thinning-only cases (67.3±6.4 μm). The complementary nature of NFL and VRH measurements in glaucoma diagnosis may be explained by the variation of NFL thickness and vessel size in the population. NFL-based diagnosis may be less sensitive in people with thicker NFL, and more sensitive in people with thinner NFL. On the other hand, vessel relief height-based diagnosis may be less sensitive in people with smaller vessels, and more sensitive in people with larger vessels. In our study population, there appeared to be little overlap between these two types. Thus, the or-logic combination of NFL and vessel relief height may greatly enhance diagnostic sensitivity with little reduction in specificity. The combination enhanced diagnostic accuracy overall, as demonstrated by increased AROC.

A slightly larger dataset of 42 patients in the study included 25 controls and 17 participants with glaucoma, all of whom completed the study without any adverse effects. In the control group, the average age was 54±10 years and 68% were female. In the glaucoma groups, the average age was 68±8, 29% were female, and the mean HVF MD was −2.48±3.73. Table 4 shows vessel relief height data of this larger dataset displayed as mean±standard deviation as a function of the radial distance from the optic disc center.

TABLE 4

Vessel Relief Height (μm)

| Radius (mm) | Control | Glaucoma | p Value |
|---|---|---|---|
| 1.2 | 15.1 ± 5.0 | 16.3 ± 10.2 | 0.778 |
| 1.9 | 13.1 ± 4.7 | 15.7 ± 9.5 | 0.505 |
| 2.6 | 9.4 ± 6.0 | 19.4 ± 15.2 | 0.007 |
| 3 | 7.1 ± 4.5 | 17.0 ± 12.2 | <0.001 |
| 3.5 | 8.4 ± 4.5 | 19.5 ± 16.9 | 0.038 |

In this dataset, the AROC value for vessel relief height (VRH) at a radius of 3.0 mm (0.85±0.07) was comparable to the AROC value for RNFL thickness (0.85±0.06) in detecting glaucoma (Table 5, showing the diagnostic power of vessel relief height measurements in detecting glaucoma). When VRH and RNFL thickness were combined, the AROC value reached 0.90±0.05 (p=0.049 vs. VRH alone; p=0.047 vs. RNFL thickness alone).

TABLE 5

| | Radius (mm) | AROC ± SEM |
|---|---|---|
| Vessel Relief | 1.2 | 0.53 ± 0.11 |
| Height | 1.9 | 0.56 ± 0.10 |
| | 2.6 | 0.75 ± 0.08 |
| | 3.0 | 0.85 ± 0.07 |
| | 3.5 | 0.69 ± 0.09 |
| RNFL Thickness | 1.7 | 0.85 ± 0.06 |
| Vessel Relief | 3.0/1.7 | 0.90 ± 0.05 |
| Height and RNFL | | (p = 0.047) |
| Thickness | | |

In the above example, the vessels were manually picked and vessel relief height was manually estimated. In other embodiments, these processes may be automated. A flow chart for a method of calculating average vessel relief height around the arcuate NFL bundle is illustrated in FIG. 10 and discussed below. The method of FIG. 10 was used to calculate vessel relief height for the dataset in the above example, and no significant differences in AROC were found between two vessel relief height calculation techniques or between the vessel relief height variable and the NFL.

Further, the above example describes an optimal radius for detecting glaucoma using retinal vessel relief height to be approximately 3.0 millimeters from the optic disc, i.e., a circular scan diameter of approximately 6.0 millimeters. For example, the radius may be in the range of 2.5 millimeters to 3.5 millimeters. Several anatomic processes may contribute to this optimal radius. For example, glaucomatous RNFL thickness change may be greatest at smaller radii from the optic disc center; therefore, a greater change in retinal vessel relief height at smaller radii may be expected. However, the results described in the above example unexpectedly showed greatest glaucomatous vessel relief height changes at a radius of 3.0 mm. Therefore, other anatomic processes are likely to be contributing to this phenomenon. One possibility is that retinal vessels near the optic disc appear more buried within the retina as the extracellular vascular support near the optic disc is lost with advancing glaucoma, which may lead to a smaller overall vessel relief height closer to the optic disc, consistent with the results described above in the example.

In the above example, the use of a high speed, swept-source OCT enabled fast, dense 3D volumetric scan acquisition to explore multiple radii to identify the optimal radius for vessel relief height measurement in glaucoma detection. However, in other examples, it may be possible to use lower-speed commercial OCT systems to make similar measurements at the optimal radius of 3 mm. For example, time-domain OCT or any other suitable OCT scanning methodologies may be used for the measurements.

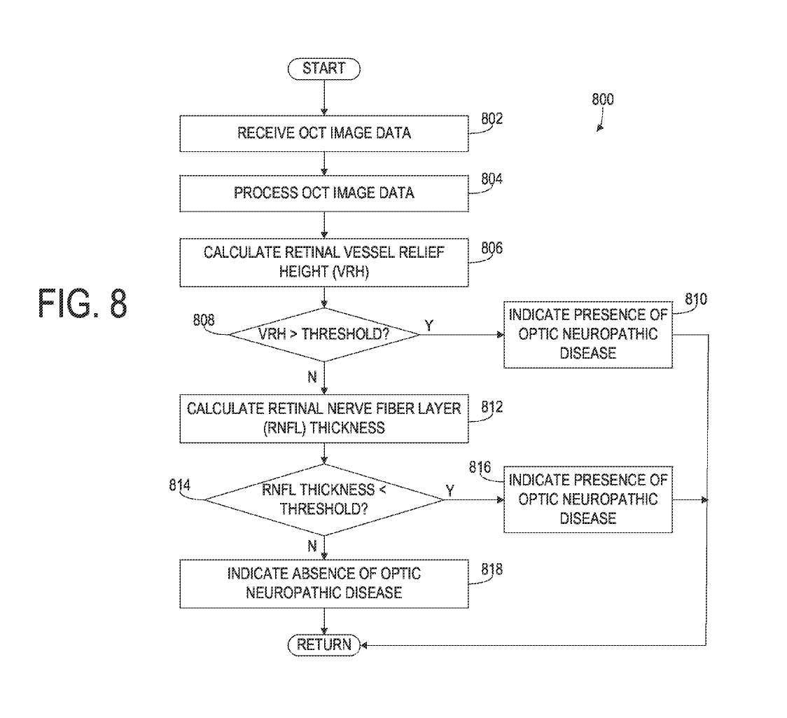

Turning now to FIG. 8, an example method 800 for detecting optic neuropathy in a subject is shown, in accordance with various embodiments. In particular, the method 800 may be used to detect and diagnose an optic neuropathic disease, such as glaucoma, in an eye of a subject based on vessel relief height measurements obtained from OCT image data. One or more steps of the method 800 may be performed via a computing system, such as the computing device 900 described below. In embodiments, one or more steps of the method 800 may be automatically performed via a computing device. Further, various acts illustrated in FIG. 8 may be performed in the sequence illustrated, in other sequences, in parallel, or in some cases omitted.

At 802, the method 800 may include receiving OCT data. For example, the OCT data may be received by a computing device from an OCT scanning system via a network or from a storage medium coupled to the computing device. The OCT data may be obtained from a circular OCT scan at a predetermined radius from the optic disc center of an eye of a subject. For example, the predetermined radius may be approximately 3.0 mm so that the circular optical tomography scan has a diameter of approximately 6.0 mm. The OCT data may be obtained from any suitable OCT scanning device, e.g., a swept-source Fourier-domain OCT scanner, a time-domain OCT scanner, or any other suitable OCT scanning technology. In particular, OCT may be used capture a circular scan around the optic disc in order to obtain a projection view of the ONH. The ONH center may be identified and selected in any suitable way. For example, the ONH center may be automatically identified and selected via a computing device or may be selected via a user input to an input device coupled to the computing device.

At 804, the method 800 may include processing the OCT image data. Various processing algorithms may be applied to the OCT data in order to condition the image data and extract parameters such as vessel relief height, vessel diameter, and nerve fiber layer thickness from the OCT data. Example processing algorithms are described in the example given above. For example, in order to reduce eye motion artifact during scanning, an orthogonal registration algorithm may be applied to register OCT scans. As another example, the boundary of inner limited membrane may be detected via a suitable boundary detection algorithm. Further, one or more retinal blood vessels may be identified and selected in the circular scan. For example, positions of one or more of the four major retinal vessels (the superior and inferior temporal arcade arteries and veins) may be identified automatically by the computing device or may be selected via a user input received via a user input device coupled to the computing device.

At 806, the method 800 may include calculating a retinal vessel relief height relative to a retinal plane from the OCT image of the eye of the subject. The retinal vessel relief height may comprise an average height relative to the retinal plane at the predetermined radius of one or more retinal blood vessels, e.g., one or more of the superior and inferior temporal arcade arteries and veins. For example, a vessel relief height may be calculated for one or more retinal vessels by calculating the number of pixels corresponding to a portion, e.g., a peak, of the vessel above the retinal plane as described above with regard to FIG. 1. The vessel relief heights of the one or more vessels may then be graded and averaged to obtain an average vessel relief height.

At 808, the method 800 may include determining if the retinal vessel relief height is greater than a retinal vessel relief height threshold. The retinal vessel relief height threshold may comprise any suitable distance threshold and may be predetermined and stored in a storage component of the computing device. The vessel relief height threshold may be based on vessel relief height data obtained from eyes of subjects who lack optic neuropathic disease. For example, the retinal vessel relief height threshold may comprise a predetermined standard deviation above the mean of vessel relief height measurements obtained from eyes of subjects who lack optic neuropathic disease, e.g., the vessel relief height threshold may comprise a 99 percentile (2.33 standard deviation above the mean of a healthy control group) cutoff for vessel relief height. Other thresholds may be used (e.g., a 95 percentile cutoff).

If the retinal vessel relief height is greater than the retinal vessel relief height threshold at 808, then method 800 proceeds to 810. At 810, the method 800 may include indicating the presence of an optic neuropathic disease in response to the retinal vessel relief height greater than the retinal vessel relief height threshold. As described above, retinal vessel relief height may be used to diagnose early stage glaucoma; thus, in some examples, the presence of an early stage optic neuropathic disease may be determined and indicated in response to determining that the retinal vessel relief height is greater than the retinal vessel relief height threshold. Indicating the presence of an optic neuropathic disease may comprise providing any suitable output via a computing device. For example, a visual indication may be output to a display device coupled to the computing device, an audio indication may be output to one or more speakers coupled to the computing device, and/or indication data may be stored in a storage medium of the computing device and/or output to an external device via a network or cable (e.g., output to a local printer for printing). Further, in some examples, a severity of the optic neuropathic disease may be indicated based on the retinal vessel relief height relative to the vessel relief height threshold. For example, if a first retinal vessel relief height is greater than the retinal vessel relief height threshold by a first amount, then a first amount of glaucoma severity may be indicated, whereas if a second retinal vessel relief height is greater than the retinal vessel relief height threshold by a second amount greater than the first amount, then a second amount of glaucoma severity greater than the first amount of glaucoma severity may be indicated. In particular, increasing vessel relief height may be correlated with increasing glaucoma severity in some examples.

However, if the retinal vessel relief height is not greater than the retinal vessel relief height threshold at 808, this condition may be used to indicate that an early stage optic neuropathic disease is not present. However, a later/advanced stage optic neuropathic disease that may not be detectable based on vessel relief height may nevertheless be present. As remarked above, vessel relief height measurements may be used to compliment OCT-derived RNFL thickness measurements in order to achieve substantial enhancement of detection sensitivity without significant loss of specificity in the diagnosis of glaucoma. Thus, when combined together, RNFL and vessel relief height measurements may enable increased sensitivity of detection of the full range of glaucoma severity. Thus, the method 800 may proceed to 812, if the retinal vessel relief height is not greater than the retinal vessel relief height threshold at 808, in order to detect the presence or absence of an optic neuropathic disease based on a retinal nerve fiber layer thickness.

At 812, the method 800 may include calculating a RNFL thickness from the OCT image in order to determine the presence or absence of an optic neuropathic disease. For example, a RNFL thickness may be obtained from a resampled volume scan of the OCT image data and may be calculated in any suitable manner. For example, RNFL thickness may be calculated based on a detected retinal ILM boundary and lower RNFL boundary, as described in the example given above.

At 814, the method 800 may include determining if the RNFL thickness is less than an RNFL thickness threshold. The RNFL thickness threshold may be based on RNFL thickness data obtained from eyes of subjects who lack optic neuropathic disease. For example, the RNFL thickness threshold may comprise a predetermined standard deviation below the mean of retinal nerve fiber layer thickness measurements obtained from eyes of subjects who lack optic neuropathic disease, e.g., the RNFL thickness threshold may comprise a 1 percentile (2.33 standard deviation below the mean of a healthy control group) cutoff value. Other thresholds may be used (e.g., a 5 percentile cutoff)

If RNFL thickness is less than the RNFL thickness threshold at 814, the method 800 proceeds to 816. At 816, the method 800 may include indicating the presence of an optic neuropathic disease in response determining that the RNFL thickness is less than the RNFL thickness threshold. As described above, RNFL thickness may be used to diagnose advanced or later stage glaucoma; thus, in some examples, the presence of an advanced stage optic neuropathic disease may be determined and indicated in response to determining that the RNFL thickness is less than the RNFL thickness threshold. Indicating the presence of an optic neuropathic disease may comprise providing any suitable output via a computing device. For example, a visual indication may be output to a display device coupled to the computing device, an audio indication may be output to one or more speakers coupled to the computing device, and/or indication data may be stored in a storage medium of the computing device and/or output to an external device via a network or cable (e.g., output to a local printer for printing). Further, in some examples, a severity of the optic neuropathic disease may be indicated based on the RNFL thickness relative to the RNFL thickness threshold. For example, if a first RNFL thickness is less than the RNFL thickness threshold by a first amount, then a first amount of glaucoma severity may be indicated, whereas if a second RNFL thickness is less than the RNFL thickness threshold by a second amount greater than the first amount, then a second amount of glaucoma severity greater than the first amount of glaucoma severity may be indicated. In particular, decreasing RNFL thickness may be correlated with increasing glaucoma severity in some examples.

However, if RNFL thickness is not less than the RNFL thickness threshold at 814, then method 800 proceeds to 818. At 818, the method 800 may include indicating the absence of an optic neuropathic disease. Indicating the absence of an optic neuropathic disease may additionally include outputting a confidence measure associated with the indication of the absence of an optic neuropathic disease. Such a confidence measure may comprise any suitable reliability or error parameter and may be based on the vessel relief height and RNFL thickness calculations. Indicating the absence of an optic neuropathic disease may comprise providing any suitable output via a computing device. For example, a visual indication may be output to a display device coupled to the computing device, an audio indication may be output to one or more speakers coupled to the computing device, and/or indication data may be stored in a storage medium of the computing device and/or output to an external device via a network or cable (e.g., output to a local printer for printing).

In some embodiments, the above described methods and processes may be tied to a computing system, including one or more computers. In particular, the methods and processes described herein, e.g., the method 800 described above and the method 1000 described below, may be implemented as a computer application, computer service, computer application program interface (API), computer library, and/or other computer program product. In some embodiments, the methods and processes described herein may be implemented by specially configured circuitry (e.g., fixed or reprogrammable circuitry), such as one or more microprocessors, application specific integrated circuits (ASICs), logic chip arrangements, and/or any other suitable circuitry.

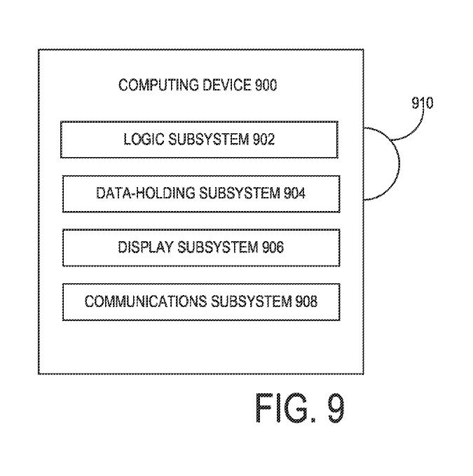

FIG. 9 schematically shows a nonlimiting computing device 900 that may perform one or more of the above described methods and processes. The computing device 900 is shown in simplified form. It is to be understood that any suitable computer architecture may be used without departing from the scope of this disclosure. In different embodiments, computing device 900 may take the form of a microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 900 includes a logic subsystem 902 and a data-holding subsystem 904. Computing device 900 may optionally include a display subsystem 906 and a communication subsystem 908, and/or other components not shown in FIG. 9. Computing device 900 may also optionally include user input devices such as manually actuated buttons, switches, keyboards, mice, game controllers, cameras, microphones, and/or touch screens, for example.

Logic subsystem 902 may include one or more physical devices configured to execute one or more machine-readable instructions. For example, the logic subsystem 902 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, and which may be remotely located and/or configured for coordinated processing. One or more aspects of the logic subsystem may be virtualized and executed by remotely accessible networked computing devices configured in a cloud computing configuration. In some embodiments, logic subsystem 902 may be implemented using specially configured circuitry, such as one or more ASICs, programmable logic arrays (PLAs), or other logic circuitry.

Data-holding subsystem 904 may include one or more physical, non-transitory, devices configured to hold data and/or instructions executable by the logic subsystem to implement the herein described methods and processes. When such methods and processes are implemented, the state of data-holding subsystem 904 may be transformed (e.g., to hold different data).

Data-holding subsystem 904 may include removable media and/or built-in devices. Data-holding subsystem 904 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 904 may include devices with one or more of the following characteristics: volatile, nonvolatile, dynamic, static, read/write, read-only, random access, sequential access, location addressable, file addressable, and content addressable. In some embodiments, logic subsystem 902 and data-holding subsystem 904 may be integrated into one or more common devices, such as an ASIC or a system on a chip.

FIG. 9 also shows an aspect of the data-holding subsystem 904 in the form of removable computer-readable storage media 910, which may be used to store and/or transfer data and/or instructions executable to implement the herein described methods and processes. Removable computer-readable storage media 910 may take the form of CDs, DVDs, HD-DVDs, Blu-Ray Discs, EEPROMs, flash memory cards, and/or floppy disks, among others.

When included, display subsystem 906 may be used to present a visual representation of data held by data-holding subsystem 904. As the herein described methods and processes change the data held by the data-holding subsystem, and thus transform the state of the data-holding subsystem 904, the state of display subsystem 906 may likewise be transformed to visually represent changes in the underlying data. Display subsystem 906 may include one or more display devices utilizing virtually any type of technology. Such display devices may be combined with logic subsystem 902 and/or data-holding subsystem 904 in a shared enclosure, or such display devices may be peripheral display devices.

When included, communication subsystem 908 may be configured to communicatively couple computing device 900 with one or more other computing devices, and/or to communicatively couple various components of computing device 900 with each other. Communication subsystem 908 may include wired and/or wireless communication devices compatible with one or more different communication protocols. As nonlimiting examples, the communication subsystem 908 may be configured for communication via a wireless telephone network, a wireless local area network, a wired local area network, a wireless wide area network, a wired wide area network, etc. In some embodiments, the communication subsystem 908 may allow computing device 900 to send and/or receive messages to and/or from other devices via a network such as the Internet.

In some embodiments, computing device 900 may be coupled to an OCT machine and may provide control signals to the OCT machine to cause the OCT machine to capture OCT images of a subject. In some embodiments, the control signals may include parameters for the operation of the OCT machine. For example, in some embodiments, the control signals may indicate a location at which a circular OCT image should be captured (e.g., a radius from a landmark, such as an optic disc center).

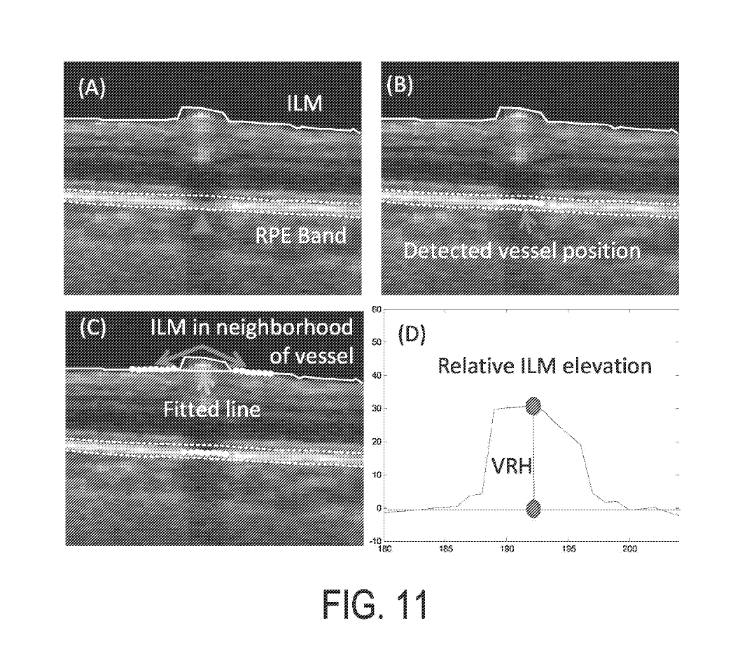

Turning now to FIG. 10, an example method 1000 for automatic vessel relief height estimation is shown, in accordance with various embodiments. In particular, the method 1000 may be used as part of a procedure for detecting and diagnosing an optic neuropathic disease, such as glaucoma, in an eye of a subject based on vessel relief height measurements obtained from OCT image data (e.g., the method of FIG. 8). One or more steps of the method 1000 may be performed via a computing system, such as the computing device 900 described above. In embodiments, one or more steps of the method 1000 may be automatically performed via a computing device. Further, various acts illustrated in FIG. 10 may be performed in the sequence illustrated, in other sequences, in parallel, or in some cases omitted. FIG. 11 shows example illustrations of various operations in the method of FIG. 10 in accordance with various embodiments.

At 1002, the method 1000 may include detecting, from OCT data, the inner limited membrane (ILM) and the retinal pigment epithelium (RPE). An example of this is illustrated in FIG. 11(A). The ILM and the RPE may be determined based on analysis of a circular OCT scan centered at the optic disc (e.g., at a radius of approximately 3.0 millimeters).

At 1004, the method 1000 may include detecting vessels by their shadows in the RPE band. In some embodiments, vessels may be detected by identifying depress points in the profile of the average intensity of the RPE band for each axial scan. An example of this is illustrated in FIG. 11(B). Any suitable image processing technique may be used.

At 1006, the method 1000 may include, for each vessel detected in 1004, calculating a relative ILM elevation for that vessel. The relative ILM elevation may be calculated as the ILM elevation of the vessel minus a local ILM elevation in a neighborhood of the vessel. The local ILM elevation may be a fitted line of ILM elevation in the neighborhood of the vessel (for example, 1-100 microns from the edge of the vessel). An example of this is illustrated in FIG. 11(C).

At 1008, the method 1000 may include, for each vessel detected in 1004, calculating a vessel relief height for that vessel. The vessel relief height may be calculated as the maximum value of the relative ILM elevation in the vessel area. An example of this is illustrated in FIG. 11(D).

At 1010, the method 1000 may include averaging the calculated vessel relief heights for a subset of the vessels detected at 1004 to generate an automated vessel relief height estimate. This subset may include those vessels in the quadrant near the arcuate bundle, and having a diameter greater than a predetermined threshold. In some embodiments, the predetermined threshold may be approximately 50 microns.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed as suitable.

The subject matter of the present disclosure includes all novel and nonobvious combinations and subcombinations of the various processes, systems and configurations, and other features, functions, acts, and/or properties disclosed herein, as well as any and all equivalents thereof.

The following paragraphs describe various example embodiments. Example 1 is a computerized method for analyzing an optical coherence tomography image of a region of an eye of a subject, including: receiving, by the computing system, optical coherence tomography image data obtained from a circular optical coherence tomography scan at a predetermined radius from an optic disc center of the eye; based on the optical coherence tomography image data, calculating, by the computing system, a retinal vessel relief height relative to a retinal plane at the predetermined radius; comparing, by the computing system, the retinal vessel relief height to a predetermined retinal vessel relief height threshold; and outputting, by the computing system, an indication of the comparison of the retinal vessel relief height to the predetermined retinal vessel relief height threshold.

Example 2 may include the subject matter of Example 1, and may further specify that, in response to the retinal vessel relief height greater than a retinal vessel relief height threshold, outputting the indication includes indicating the presence of an optic neuropathic disease condition.

Example 3 may include the subject matter of Example 2, and may further specify that the optic neuropathic disease condition includes a glaucoma condition.

Example 4 may include the subject matter of any of Examples 1-3, and may further specify that the predetermined radius is approximately 3.0 mm.

Example 5 may include the subject matter of any of Examples 1-4, and may further specify that the retinal vessel relief height includes an average vessel relief height relative to the retinal plane at the predetermined radius of one or more retinal blood vessels.

Example 6 may include the subject matter of Example 5, and may further include: for each of the one or more retinal blood vessels, calculating, based on the optical coherence tomography image data, a relief height of that vessel relative to a retinal plane at the predetermined radius from; and averaging the relief heights of the one or more retinal blood vessels to obtain the average vessel relief height.

Example 7 may include the subject matter of any of Examples 5-6, and may further specify that the one or more retinal blood vessels includes one or more of the superior and inferior temporal arcade arteries and veins.

Example 8 may include the subject matter of any of Examples 1-7, and may further specify that, in response to the retinal vessel relief height greater than the retinal vessel relief height threshold, outputting the indication includes indicating an optic neuropathic disease severity based on the retinal vessel relief height, where increasing optic neuropathic disease severity is correlated with increasing retinal vessel relief height.

Example 9 may include the subject matter of any of Examples 1-8, and may further specify that the retinal vessel relief height threshold is based on vessel relief height measurements obtained from eyes of subjects who lack optic neuropathic disease.

Example 10 may include the subject matter of Example 9, and may further specify that the retinal vessel relief height threshold includes a predetermined standard deviation above the mean of vessel relief height measurements obtained from eyes of subjects who lack optic neuropathic disease.

Example 11 may include the subject matter of any of Examples 1-10, and may further include, in response to the retinal vessel relief height less than or equal to the retinal vessel relief height threshold, calculating a retinal nerve fiber layer thickness from the optical coherence tomography image data and determining the presence or absence of an optic neuropathic disease based at least in part on the retinal nerve fiber layer thickness.

Example 12 may include the subject matter of Example 11, and may further specify that outputting the indication includes outputting an indication of the presence of an early stage optic neuropathic disease in response to the retinal vessel relief height greater than the retinal vessel relief height threshold and outputting an indication of the presence of an advanced stage optic neuropathic disease in response to the retinal nerve fiber layer thickness less than a retinal nerve fiber layer thickness threshold.

Example 13 may include the subject matter of Example 12, and may further specify that the retinal nerve fiber layer thickness threshold includes a predetermined standard deviation below the mean of retinal nerve fiber layer thickness measurements obtained from eyes of subjects who lack optic neuropathic disease.

Example 14 is a computerized method for detecting glaucoma in an eye of a subject, including: receiving, by a computing system, optical coherence tomography image data obtained from a circular optical coherence tomography scan at a radius of approximately 3.0 mm from the optic disc center of the eye; based on the optical coherence tomography image data, calculating, by the computing system, a retinal vessel relief height relative to a retinal plane at the radius; and indicating, by the computing system, the presence of a glaucoma condition in response to a determination that the retinal vessel relief height is greater than a retinal vessel relief height threshold.

Example 15 may include the subject matter of Example 14, and may further include, in response to a determination that the retinal vessel relief height is less than or equal to the retinal vessel relief height threshold, calculating, by the computing system, a retinal nerve fiber layer thickness from the optical coherence tomography image data and indicating, by the computing system, the presence of a glaucoma condition in response to a determination that the retinal nerve fiber layer thickness is less than a retinal nerve fiber layer thickness threshold.

Example 16 is a computing system, including: a logic subsystem; and a data holding subsystem comprising machine-readable instructions stored thereon that are executable by the logic subsystem to: receive optical coherence tomography image data obtained from a circular optical coherence tomography scan at a predetermined radius from the optic disc center of an eye of a subject; calculate a retinal vessel relief height relative to a retinal plane at the predetermined radius from the optical coherence tomography image data; in response to a determination that the retinal vessel relief height is greater than a retinal vessel relief height threshold, output an indication of the presence of a glaucoma condition; and in response to a determination that the retinal vessel relief height is less than or equal to the retinal vessel relief height threshold: calculate a retinal nerve fiber layer thickness from the optical coherence tomography image data; in response to a determination that the retinal nerve fiber layer thickness is less than a retinal nerve fiber layer thickness threshold, output an indication of the presence of a glaucoma condition; and in response to a determination that the retinal nerve fiber layer thickness is greater than or substantially equal to the retinal nerve fiber layer thickness threshold, output an indication of the absence of a glaucoma condition.

Example 17 may include the subject matter of Example 16, and may further specify that the predetermined radius is approximately 3.0 mm.

Example 18 may include the subject matter of Example 16, and may further specify that calculate a retinal vessel relief height relative to a retinal plane at the predetermined radius from the optical coherence tomography image data includes: for each of a plurality of vessels, calculate a difference between an inner limited membrane elevation for the vessel and a local inner limited membrane elevation in the neighborhood of the vessel, and identify a maximum value of the difference; and average the maximum values associated with each of the plurality of vessels to determine the vessel relief height.

Example 19 may include the subject matter of Example 18, and may further specify that each vessel of the plurality of vessels is in a quadrant near the arcuate bundle.

Example 20 may include the subject matter of any of Examples 18-19, and may further specify that each vessel of the plurality of vessels has a diameter greater than or equal to approximately 50 microns.

Example 21 is one or more machine-readable media having instructions thereon that, in response to execution by one or more processing devices of a computing system, cause the computing system to perform the method of any of Examples 1-15.

Example 22 is a system for processing optical coherence tomography image data representative of an eye of a subject, comprising means for performing the method of any of Examples 1-15.

Example 23 is a computer system for analyzing an optical coherence tomography image of a region of an eye of a subject, including: circuitry to receive optical coherence tomography image data obtained from a circular optical coherence tomography scan at a predetermined radius from an optic disc center of the eye; circuitry to calculate, based on the optical coherence tomography image data, a retinal vessel relief height relative to a retinal plane at the predetermined radius; circuitry to compare the retinal vessel relief height to a predetermined retinal vessel relief height threshold; and circuitry to output an indication of the comparison of the retinal vessel relief height to the predetermined retinal vessel relief height threshold.

Example 24 is a computer system for detecting glaucoma in an eye of a subject, including: circuitry to receive optical coherence tomography image data obtained from a circular optical coherence tomography scan at a radius of approximately 3.0 mm from the optic disc center of the eye; circuitry to calculate, based on the optical coherence tomography image data, a retinal vessel relief height relative to a retinal plane at the radius; and circuitry to indicate the presence of a glaucoma condition in response to a determination that the retinal vessel relief height is greater than a retinal vessel relief height threshold.

1. A computerized method for analyzing an optical coherence tomography image of a region of an eye of a subject, comprising:
receiving, by a computing system, optical coherence tomography image data obtained from a circular optical coherence tomography scan at a predetermined radius from an optic disc center of the eye;
based on the optical coherence tomography image data, calculating, by the computing system, a retinal vessel relief height relative to a retinal plane at the predetermined radius;
comparing, by the computing system, the retinal vessel relief height to a predetermined retinal vessel relief height threshold; and
outputting, by the computing system, an indication of the comparison of the retinal vessel relief height to the predetermined retinal vessel relief height threshold.

2. The method of claim 1, wherein, in response to the retinal vessel relief height greater than a retinal vessel relief height threshold, outputting the indication comprises indicating the presence of an optic neuropathic disease condition.

3. The method of claim 2, wherein the optic neuropathic disease condition comprises a glaucoma condition.

4. The method of claim 1, wherein the predetermined radius is approximately 3.0 mm.

5. The method of claim 1, wherein the retinal vessel relief height comprises an average vessel relief height relative to the retinal plane at the predetermined radius of one or more retinal blood vessels.

6. The method of claim 5, further comprising:
for each of the one or more retinal blood vessels, calculating, based on the optical coherence tomography image data by the computing system, a relief height of that vessel relative to a retinal plane at the predetermined radius from; and
averaging, by the computing system, the relief heights of the one or more retinal blood vessels to obtain the average vessel relief height.

7. The method of claim 5, wherein the one or more retinal blood vessels comprises one or more of the superior and inferior temporal arcade arteries and veins.

8. The method of claim 1, wherein, in response to the retinal vessel relief height greater than the retinal vessel relief height threshold, outputting the indication comprises indicating an optic neuropathic disease severity based on the retinal vessel relief height, where increasing optic neuropathic disease severity is associated with increasing retinal vessel relief height.

9. The method of claim 1, wherein the retinal vessel relief height threshold is based on vessel relief height measurements obtained from eyes of subjects who lack optic neuropathic disease.

10. The method of claim 9, wherein the retinal vessel relief height threshold comprises a predetermined standard deviation above the mean of vessel relief height measurements obtained from eyes of subjects who lack optic neuropathic disease.

11. The method of claim 1, further comprising, in response to the retinal vessel relief height less than or equal to the retinal vessel relief height threshold, calculating, by the computing system, a retinal nerve fiber layer thickness from the optical coherence tomography image data and determining the presence or absence of an optic neuropathic disease based at least in part on the retinal nerve fiber layer thickness.

12. The method of claim 11, wherein outputting the indication comprises outputting an indication of the presence of an early stage optic neuropathic disease in response to the retinal vessel relief height greater than the retinal vessel relief height threshold and outputting an indication of the presence of an advanced stage optic neuropathic disease in response to the retinal nerve fiber layer thickness less than a retinal nerve fiber layer thickness threshold.

13. The method of claim 12, wherein the retinal nerve fiber layer thickness threshold comprises a predetermined standard deviation below the mean of retinal nerve fiber layer thickness measurements obtained from eyes of subjects who lack optic neuropathic disease.

14. A computerized method for detecting glaucoma in an eye of a subject, comprising:
receiving, by a computing system, optical coherence tomography image data obtained from a circular optical coherence tomography scan at a radius of approximately 3.0 mm from the optic disc center of the eye;
based on the optical coherence tomography image data, calculating, by the computing system, a retinal vessel relief height relative to a retinal plane at the radius; and
indicating, by the computing system, the presence of a glaucoma condition in response to a determination that the retinal vessel relief height is greater than a retinal vessel relief height threshold.

15. The method of claim 14, further comprising, in response to a determination that the retinal vessel relief height is less than or equal to the retinal vessel relief height threshold, calculating, by the computing system, a retinal nerve fiber layer thickness from the optical coherence tomography image data and indicating, by the computing system, the presence of a glaucoma condition in response to a determination that the retinal nerve fiber layer thickness is less than a retinal nerve fiber layer thickness threshold.

16. A computing system, comprising:
a logic subsystem; and
a data holding subsystem comprising machine-readable instructions stored thereon that are executable by the logic subsystem to:
receive optical coherence tomography image data obtained from a circular optical coherence tomography scan at a predetermined radius from the optic disc center of an eye of a subject;
calculate a retinal vessel relief height relative to a retinal plane at the predetermined radius from the optical coherence tomography image data;
in response to a determination that the retinal vessel relief height is greater than a retinal vessel relief height threshold, output an indication of the presence of a glaucoma condition; and
in response to a determination that the retinal vessel relief height is less than or equal to the retinal vessel relief height threshold:
calculate a retinal nerve fiber layer thickness from the optical coherence tomography image data;
in response to a determination that the retinal nerve fiber layer thickness is less than a retinal nerve fiber layer thickness threshold, output an indication of the presence of a glaucoma condition; and
in response to a determination that the retinal nerve fiber layer thickness is greater than or substantially equal to the retinal nerve fiber layer thickness threshold, output an indication of the absence of a glaucoma condition.

17. The system of claim 16, wherein the predetermined radius is approximately 3.0 mm.

18. The system of claim 16, wherein calculate a retinal vessel relief height relative to a retinal plane at the predetermined radius from the optical coherence tomography image data comprises:
for each of a plurality of vessels;
calculate a difference between an inner limited membrane elevation for the vessel and a local inner limited membrane elevation in the neighborhood of the vessel, and
identify a maximum value of the difference; and
average the maximum values associated with each of the plurality of vessels to determine the vessel relief height.

19. The system of claim 18, wherein each vessel of the plurality of vessels is in a quadrant near the arcuate bundle.

20. The system of claim 18, wherein each vessel of the plurality of vessels has a diameter greater than or equal to approximately 50 microns.

21. One or more machine-readable media having instructions thereon that, in response to execution by one or more processing devices of a computing system, cause the computing system to perform the method of claim 1.

22. A system for processing optical coherence tomography image data representative of an eye of a subject, comprising means for performing the method of claim 1.
